(19) United States
(12) Patent Application Publication
(10) Pub. No.: US 2006/0051778 A1
(43) Pub. Date: Mar. 9, 2006

(54) Diagnosis of systemic lupus erythematosus

(76) Inventor: Charles A. Kallick, Lemont, IL (US)

Correspondence Address: WELSH & KATZ, LTD, 120 S RIVERSIDE PLAZA, 22ND FLOOR, CHICAGO, IL, 60606, US

(21) Appl. No.: 11/093,805

(22) Filed: Mar. 30, 2005

Related U.S. Application Data
(60) Provisional application No. 60/557,947, filed on Mar. 31, 2004.

(51) Int. Cl. C12Q 1/68; G01N 33/554; G01N 33/569

(52) U.S. Cl. 435/6; 435/7.32

(57) ABSTRACT

Diagnostic methods for the detection of SLE in a human body sample are disclosed. Nucleic acid hybridization and antibody-based methods derived from identification of Mycoplasma haemosapiens or its 16S sequence are described.

### CROSS-REFERENCE TO RELATED APPLICATION

This application claims priority from U.S. Ser. No. 60/557,947 filed Mar. 31, 2004.

### TECHNICAL FIELD

This invention relates to the diagnosis of systemic lupus erythematosus. More particularly, this invention relates to methods for the detection of Mycoplasma haemosapiens in a patient.

### BACKGROUND OF THE INVENTION

Systemic lupus erythematosus (SLE) is severe disease characterized in most patients by a chronic inflammation (swelling, redness, and pain). SLE affects multiple systems in the body that include skin, joints, blood, lungs, kidneys, heart, brain, gastrointestinal tract, liver, and nervous system. Patients having this disease produce antibodies in their blood that target cells of various body tissues. These antibody-targeted cells are then destroyed or injured by their own white blood cell mediated injury causing cell death, inflammation, and pain. As such, SLE is known as an autoimmune disease where one's own immune system attacks rather than protects the body.

No etiological agent has yet been found that qualifies as he exciting exogenous agent believed to cause the cascade of events comprising the disease SLE, although the search has been extensive. Crow et al., “Etiologic Hypothesis for Systemic Lupus Erythematosus,” in Lahita Systemic Lupus Erythematosus, Churchill, Livingston, N.Y. (1987) page 51 ff. There is general agreement that tissue and organ injury in SLE is mediated by immune phenomena. Unexplained at this time is the predilection of SLE for females. Taurog et al., Intern. J. Derm., 20:149-158 (1981).

Many viral etiologic agents have been sought although none have been convincingly demonstrated. Pincus, Arthr. Rheum., 25:847 (1982). More recently, characterizations of soluble products of bacteria and mycoplasmas with unique capacities to perturb immune systems have led to new considerations in regard to the infectious trigger of SLE.

For example, intra-erythrocyte organisms with characteristics like the Anaplasmataceae that were thought to be Haemobartonella-like were first suggested as exogenous exciting agents in SLE by Kallick et al., Nature New Biology, 236:145-146 (1972). The Anaplasmataceae family of bacteria were Proteobacteria of the order Rickettsiales. That report was further developed by a later report of antigenic similarities between SLE or lupus nephritis and diseases caused by Anaplasma marginale, an intra-erythrocytic parasite of cattle and a member of the family Anaplasmataceae (at that time) Kallick et al., Arthr. Rheum., 23:197-205 (1980).

Further, exogenous intra-erythrocytic structures seen in the same erythrocyte by giemsa staining and phase contrast microscopy which were identical or similar in appearance to Mycoplasma haemofelis, the causative agent of feline infectious anemia, have been observed in most patients with SLE, and are illustrated in U.S. Pat. No. 5,972,309 and U.S. Pat. No. 5,795,563.

The Anaplasmataceae were a descriptive classification of hemotropic bacteria based on morphologic characteristics and included organisms now recognized as unrelated through relationships and biologic characteristics defined by 16S ribosomal RNA (rRNA). Anaplasma marginale, the causative agent of bovine Anaplasmosis, although very similar in morphology and characteristics, has been shown to be an Ehrlichia. Haemobartonella haemofelis, the causative agent of feline infectious anemia has been shown to be a bacteria belonging to the genus Mycoplasma, and now is identified as Mycoplasma haemofelis. Similarly, Haemobartonella haemocanis, that causes an obscure infection of dogs, is renamed Mycoplasma haemocanis, and Eperythrozoon suis that causes a disease in pigs is now Mycoplasma haemosuis. Haemobartonella muris remains the same but should soon be reclassified as a Mycoplasma as the others have been.

On the presumption of Anaplasmataceae parasitemia, several humans with SLE have been treated with tetracycline or doxycycline, a tetracycline related drug as in the commonly accepted treatment for Anaplasmataceae infection. Three patients treated by one of us are exemplary.

The first was a 17 year old female with severe SLE and nephritis who experienced a lysis of fever within a week of therapy with disappearance of Haemobartonella-like agents from the circulating erythrocytes as observed by acridine orange and fluorescent antibody determination. This patient was not subsequently followed.

The second patient is a male with SLE who has been taking tetracycline for his lupus for 10 years. He stated that his fever, joint pains, and other symptoms disappeared while he was taking tetracycline. He had first been given the antibiotic for treatment of another infection and noted it caused amelioration of his SLE.

The third is a patient who has mixed connective tissue disorder resembling SLE but with a negative ANA titer. This patient went into remission of her symptomology after 3 weeks of therapy with tetracycline and had remained in clinical remission for the subsequent 3 months. It is of interest that in addition to marked subjective improvement of this last patient, C-reactive protein became negative after tetracycline therapy was begun.

Subsequent to these initially studied patients, a large number of other patients with SLE have received continuous therapy with tetracycline or its derivatives. These treatments have been on a compassionate basis by the patients' own physicians, or as part of a study approved by an institutional review board, but not completed. Most of such treatments have resulted in amelioration of the disease state, with complete remission, or a trend in such amelioration. That study, done at Cook County Hospital, Chicago, Ill., was terminated before the results, as analyzed, were shown to be statistically significant. The negative results appeared to be based on the small numbers analyzed.

In the 1940's, there was some success in treating patients with arthritis who also had lupus with Aureomycin. Aureomycin is a tetracycline-like drug that had been proposed as a treatment for rheumatoid diseases. [Brown et al., J. Lab. Clin. Med., 34:1404-1410 (1949); and Scheff et al., Infec. Dis., 98:113 (1956).]

An alternate therapy, splenectomy, is a rare treatment for the thrombocytopenia seen in some patients with SLE. Coon, So. Amer. J. Surgery, 155:391 (1988). The spleen is regulatory in removing nuclear remnants and particles from erythrocytes. With anaplasmosis as well as malaria, splenectomy causes a new outbreak or recrudescence of the disease.

However, one splenectomy patient was found to have parasitemias of erythrocytes with intra-erythrocytic phase contrast-visible retractile bodies in up to about 16 percent of her studied erythrocytes. The intra-erythrocytic bodies were very similar in morphology to the animal hemotropic Mycoplasmas such as Mycoplasma haemofelis, Mycoplasma haemocanis and Haemobartenella muris.

A study of cats in 1896 by Howell proved the existence of intra-erythrocytic bodies. The prevalence of these bodies (now bearing the name Howell-Jolly bodies) was enhanced within a few hours or days of splenectomy. The elegant drawings of Dr. Howell, whose findings have been subsequently confirmed by others with modern methods including electron microscopy, have amply demonstrated this phenomenon. Howell-Jolly bodies are described as about 1 in diameter in an eccentric position in the erythrocyte and appear to differ from the above-noted intra-erythrocytic phase contrast-visible refractile bodies.

The present therapy of SLE is based upon the use of steroids with immunosuppressive drugs and/or plasmaphoresis (blood plasma filtering). It is of interest that Anaplasmataceae infections in animals are ameliorated by steroids, which is unique among infectious diseases. [Scheff et al., Infec. Dis., 98:113 (1956); and Ristic et al., J. Vet. Res., 19:37 (1958).] No present therapy is satisfactory in humans. The ravages of therapeutic side effects and the constant fatigue take a severe toll in well-being, general health, and increased morbidity and mortality of the estimated 500,000 Americans with this disease. [Dubois, Lupus Erythematosus, 2nd Ed., U.S. California Press, Los Angeles (1974).]

### BRIEF SUMMARY OF THE INVENTION

The present invention relates to the detection of Mycoplasma haemosapiens in a human body sample, such as whole blood, red blood cells, marrow or liver and to the diagnosis and tracking of the treatment of systemic lupus erythematosus in a human.

Thus, one aspect of the method comprises determining the presence in that human body sample of a sequence of the DNA that encodes all or part of the Mycoplasma haemosapiens 16S rRNA having SEQ ID NO:1 or a sequence complementary to that DNA sequence.

The Mycoplasma haemosapiens organism was found by polymerase chain reaction (PCR) from a splenectomized human patient who had 3.8 percent of her erythrocytes infected with the organism similar to the animal hemotropic mycoplasmas, Anaplasmataceae and Haemobartonella-like organisms described before. The new organism is identified by its rRNA sequences as being closest to Mycoplasma haemofelis or Mycoplasma haemocanis, the causative agents of feline infectious anemia and an obscure infection of dogs, respectively.

Because of the finding in a splenectomized patient with SLE and because the new organism morphologically resembles the organism seen in other patients with SLE, we identify this organism as the causative agent of SLE, and believe it is the likely cause of several other syndromes identified in the literature as autoimmune disorders.

This organism is novel. We have chosen to name it Mycoplasma haemosapiens. The human medical tests derived from the sequence of the DNA that encodes the 16S rRNA (SEQ ID NO:1) and primers designed for its amplification provide diagnostic methods for detecting this agent.

The present invention also contemplates antibody-based methods derived from identification of Mycoplasma haemosapiens or its 16S rRNA sequence. Thus, a further aspect of the invention contemplates a method of detecting the presence of Mycoplasma haemosapiens in a human body sample. One aspect of this method comprises the step of contacting a human body sample that may contain Mycoplasma haemosapiens with antibodies raised to Mycoplasma haemocanis in dogs, Mycoplasma haemofelis in cats, or Mycoplasma haemosuis in swine, or Haemobartonella muris in mice and determining whether an antibody recognition event (specific antibody binding) occurs. The occurrence of an antibody recognition event indicates the presence of Mycoplasma haemosapiens in the human body sample. Alternatively, antibodies (serum, plasma or other blood-based sample) from the patient can be contacted with a Mycoplasma or Haemobartonella antigen from one of the above-described animals that is infected with a recited Mycoplasma or Haemobartonella, and the presence of specific antibody binding determined to indicate the presence of Mycoplasma haemosapiens infection in the patient.

An above assay can be used for finding this infection in humans before, after or during the clinical manifestation of infection with this organism. In addition, optical methods utilizing giemsa stain, Wright's stains, and acridine orange stain, optionally with light refraction, are useful in identifying the intra-erythrocyte structures indicative of Mycoplasma haemosapiens infection in a human body sample, which distinguishes the structures from the previously-known Heinz bodies [John W. Adamson, Harrison's Textbook of Medicine, 15th Edition, volume 1, McGraw-Hill, New York, p. 671 (2001)] and nuclear remnants. However, those intra-erythrocyte structures are seen only in erythrocytes and are only rarely seen in those cells (a few percent of the cells of splenectomized patients). Still further, those structures are indicative but not definitive of a Mycoplasma haemosapiens infection, whereas use of the nucleic acid or antibody assays described herein can be definitive.

### BRIEF DESCRIPTION OF THE DRAWINGS

In the drawings forming a part of this disclosure,

FIG. 1 is a partial sequence of the DNA that encodes the 16S rRNA of Mycoplasma haemosapiens of SEQ ID NO: 1.

FIG. 2 Shows the forward primer (SEQ ID NO: 2) and related primer DNA sequences from Mycoplasma haemosuis, Mycoplasma haemofelis, Mycoplasma haemocanis (SEQ ID NOs: 4, 5 and 6) and E. coli (SEQ ID NO: 7) for comparison, as well as the reverse primer used herein, SEQ ID NO: 3 (shown in 3′ to 5′ direction), and sequences from the above organisms to which the reverse primer binds (SEQ ID Nos: 8, 9 and 10 respectively) and the reverse comparative sequence from E. coli (SEQ ID NO: 11). Binding regions (or regions of identity or homology) are shown in bold.

FIG. 3 is a schematic depiction of hematopoietic cell differentiation in which HSC=Human Stem Cell, CMP=myelomocytic progenitor, CLP=Lymphoid progenitor, GMP=granulocyte-monocyte progenitor, MEP=erythrocyte-megakaryocyte progenitor, and ProT=Lymphocyte T cell progenitor, and ProB=lymphocyte B cell progenitor.

FIG. 4, shown in three sheets as FIG. 4A, FIG. 4B and FIG. 4C, is a sequence of the DNA that encodes the 16S rRNA of Mycoplasma haemocanis of SEQ ID NO: 12.

FIG. 5, shown in three sheets as FIG. 5A, FIG. 5B and FIG. 5C, is a sequence of the DNA that encodes the 16S rRNA of Mycoplasma haemofelis of SEQ ID NO: 13.

FIG. 6, shown in three sheets as FIG. 6A, FIG. 6B and FIG. 6C, is a sequence of the DNA that encodes the 16S rRNA of Mycoplasma haemofelis of SEQ ID NO: 14.

FIG. 7, shown in three sheets as FIG. 7A, FIG. 7B and FIG. 7C, is a sequence of the DNA that encodes the 16S rRNA of Mycoplasma haemomuris of SEQ ID NO: 15.

### DETAILED DESCRIPTION OF THE INVENTION

The present invention contemplates the diagnosis of systemic lupus erythematosus in a human. The diagnosis is accomplished by the detection of Mycoplasma haemosapiens in a human body sample. In one aspect, the detection of Mycoplasma haemosapiens in the human patient comprises determining the presence of DNA that encodes all or part of the Mycoplasma haemosapiens 16S rRNA having SEQ ID NO:1 or a sequence complementary to that DNA sequence in a human body sample. The human body sample can be skin, joints, blood, lungs, kidneys, heart, brain, saliva, gastrointestinal tract, bone marrow, liver, and nervous system. Preferably, the human body sample is blood. For testing of patients that have not previously undergone splenectomy, Mycoplasma haemosapiens 16S polynucleotide is more evident from marrow or liver samples than from blood samples because the spleen tends to rid the circulating blood of the infected red blood cells.

Use of an isolated and purified nucleic acid having a sequence of SEQ ID NO: 1, a sequence complementary to that nucleic acid, a hybrid of that nucleic acid and its complementary sequence, and mixtures thereof are contemplated herein. Thus, the use of single stranded nucleic acid of SEQ ID NO: 1, its single stranded complement, as well as a double stranded molecule formed by hybridization of those two strands, and mixtures of those single and double stranded molecules are contemplated. Similarly, an isolated and purified individual nucleic acid having a sequence of SEQ ID NOs: 2 or 3, a sequence complementary to either of those nucleic acids, a hybrid of either nucleic acid and its complementary sequence, and mixtures of the single and double stranded molecules of each of SEQ ID NOs: 2 and 3 are contemplated herein. Additionally, those sequences can be DNA or RNA or mixtures of both, and thymine or uracil can be present bonded to ribose or deoxyribose in any of the sequences noted above.

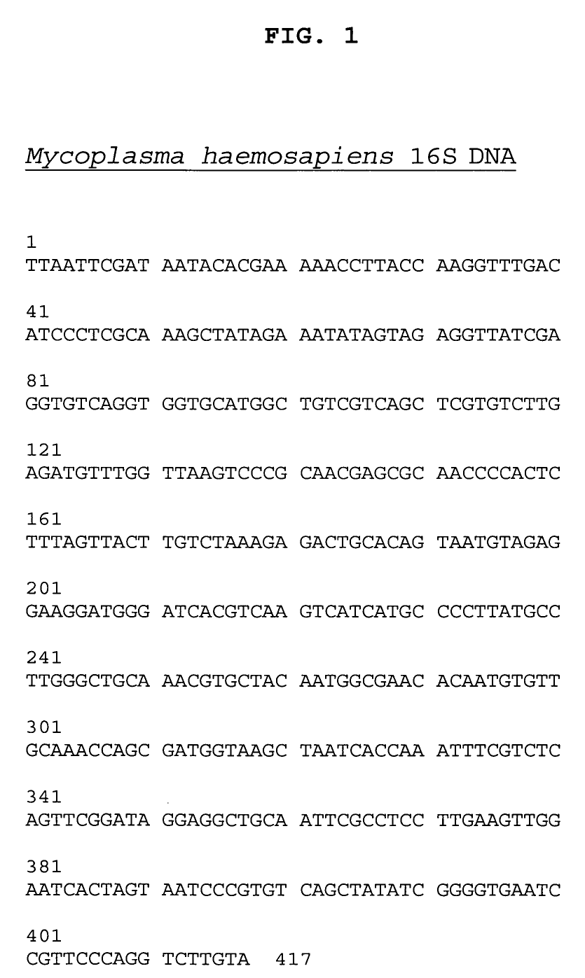

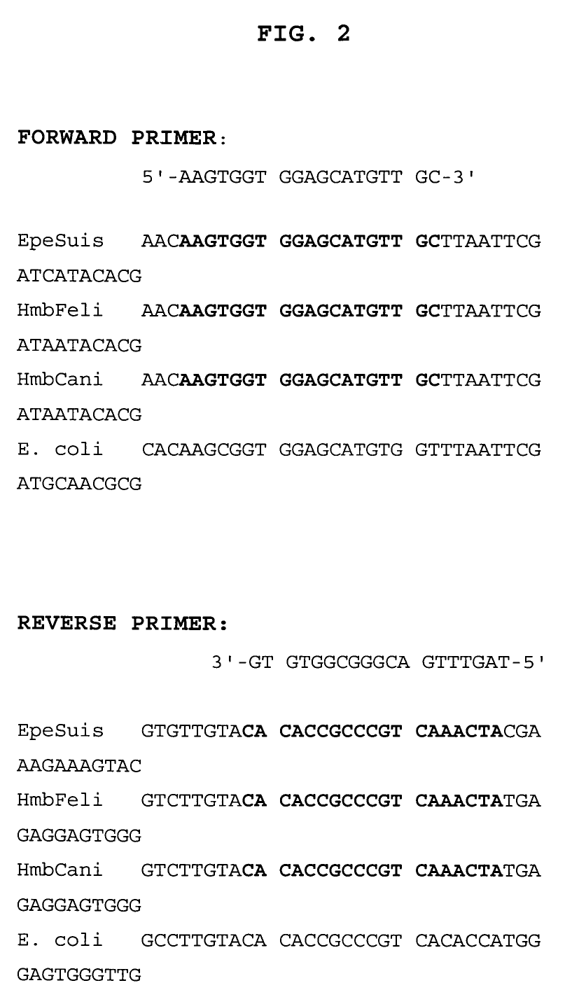

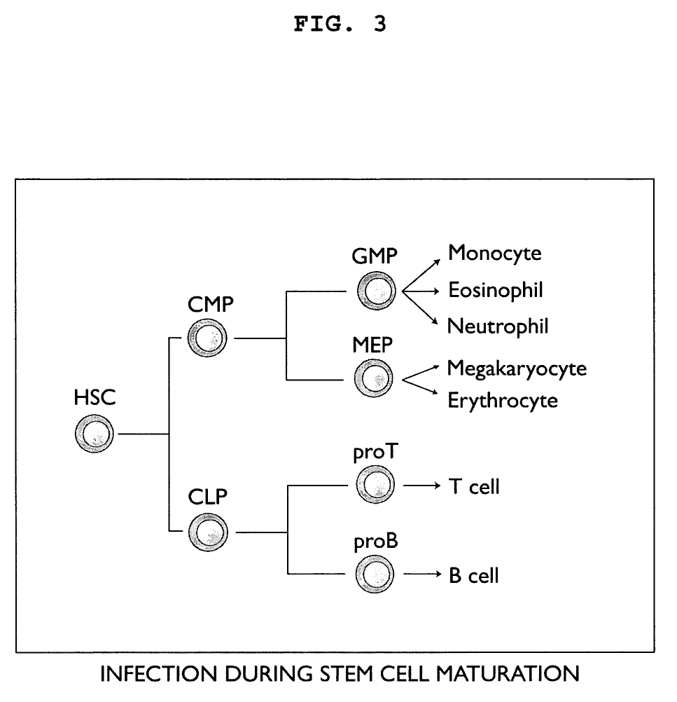

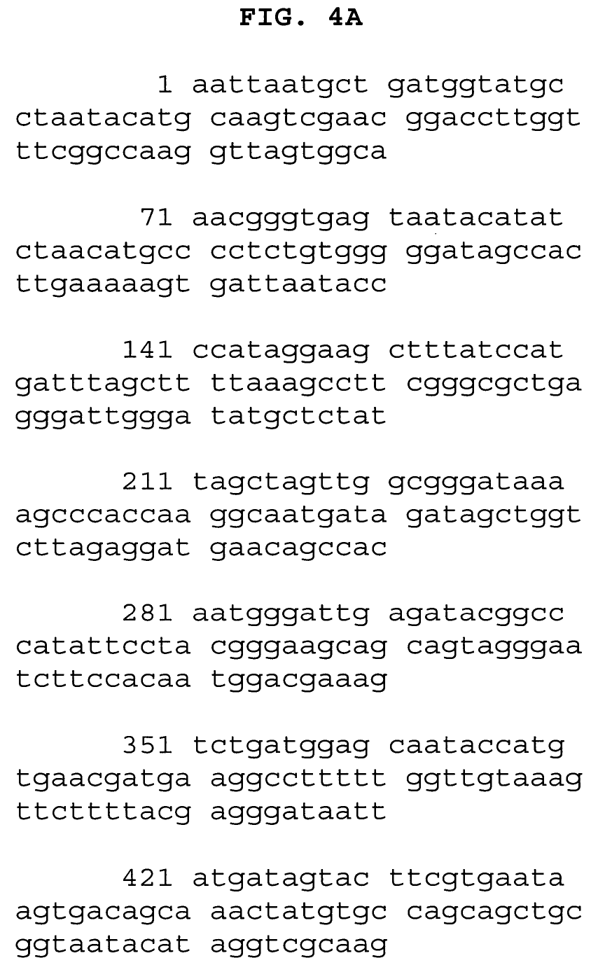

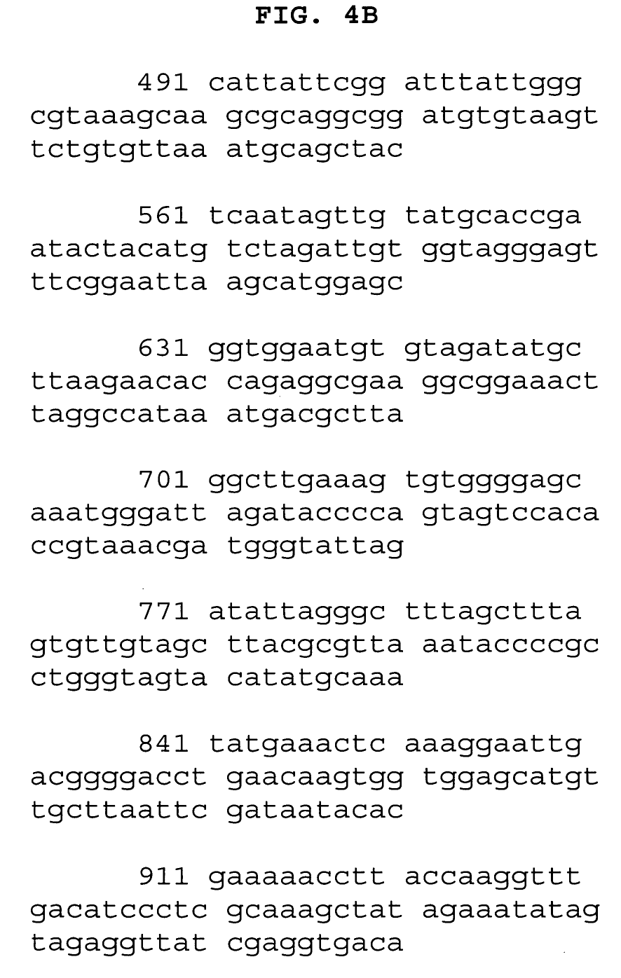

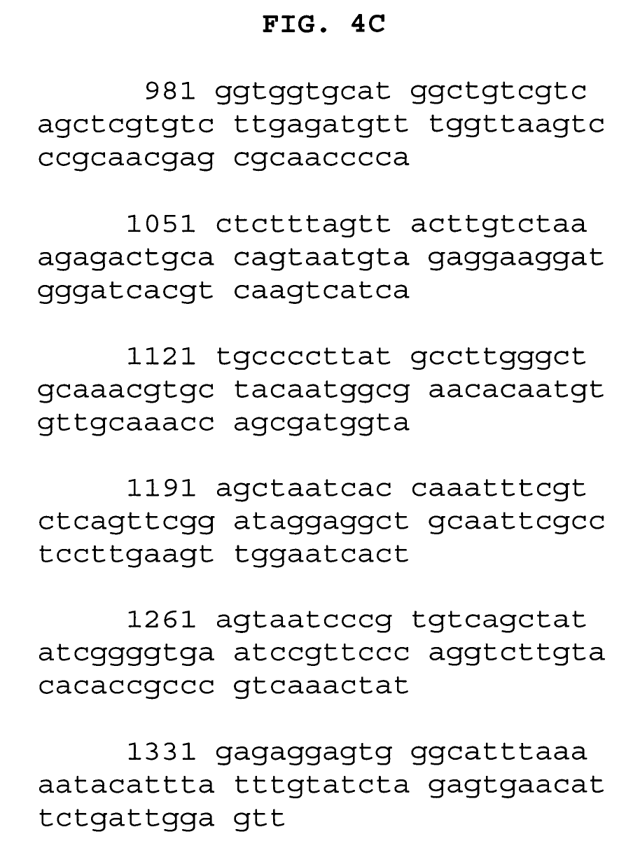

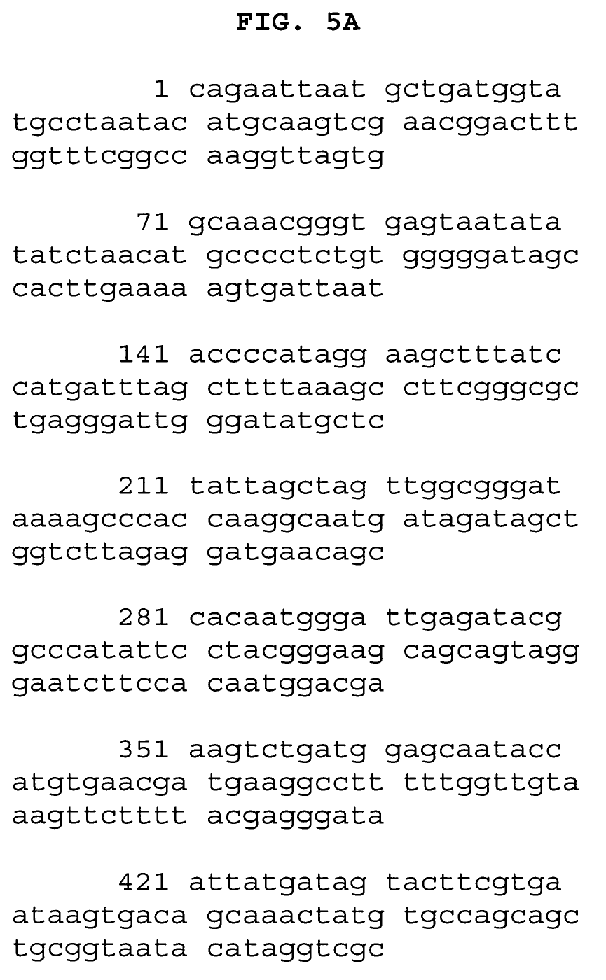

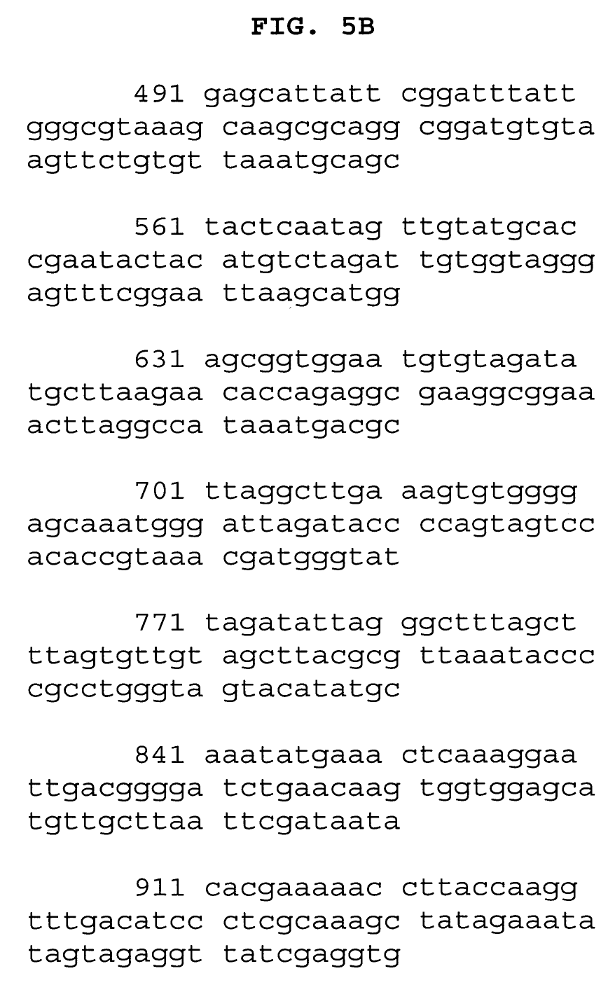

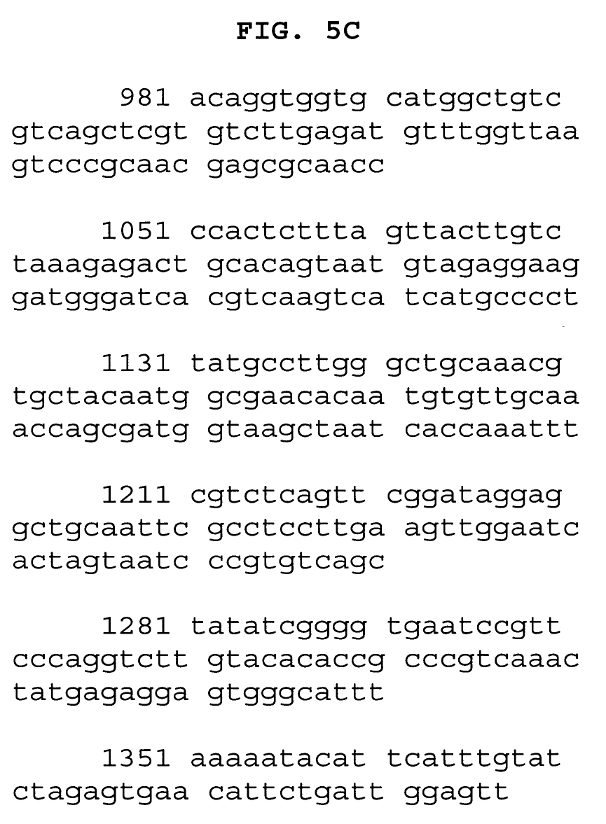

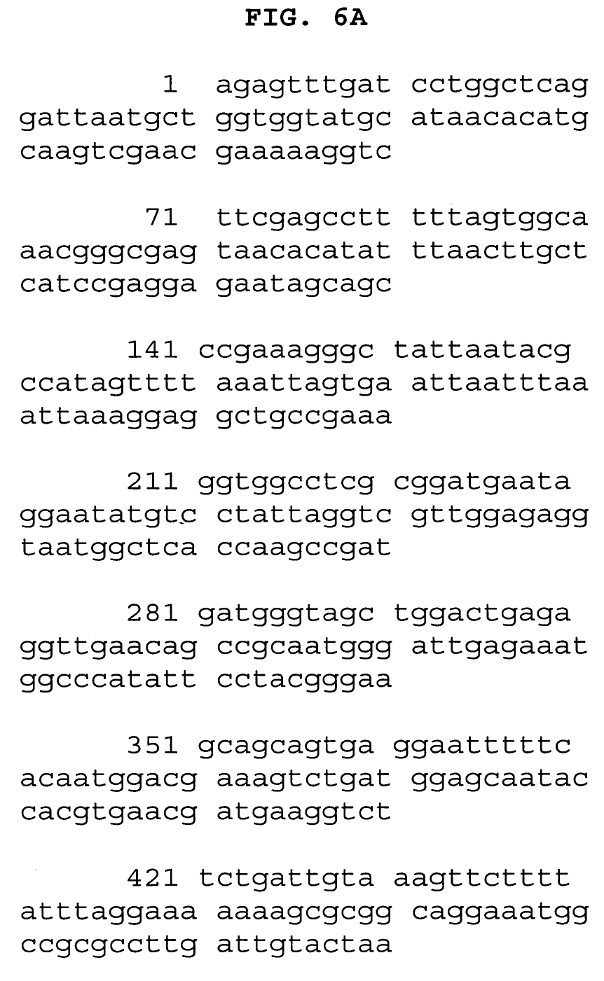

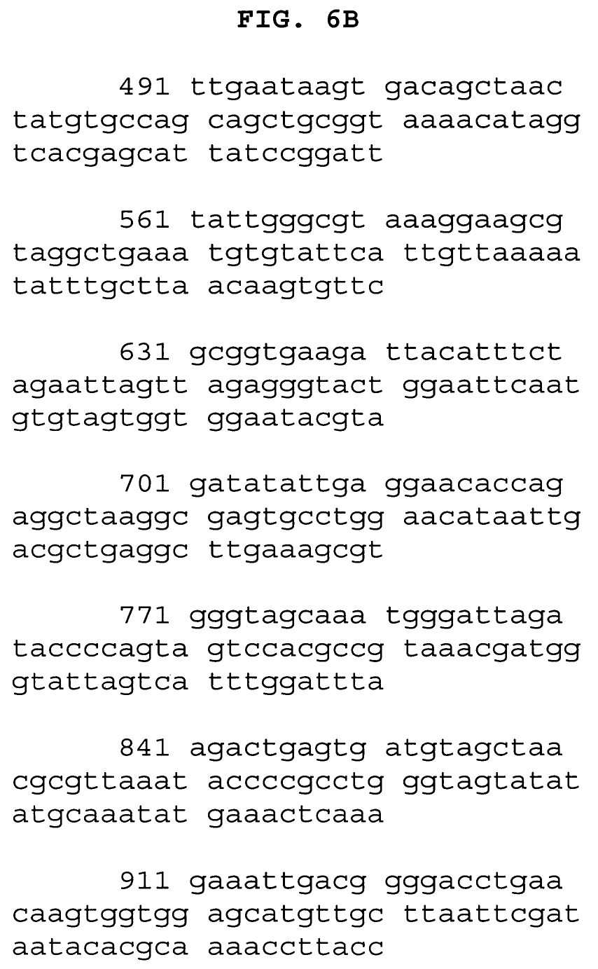

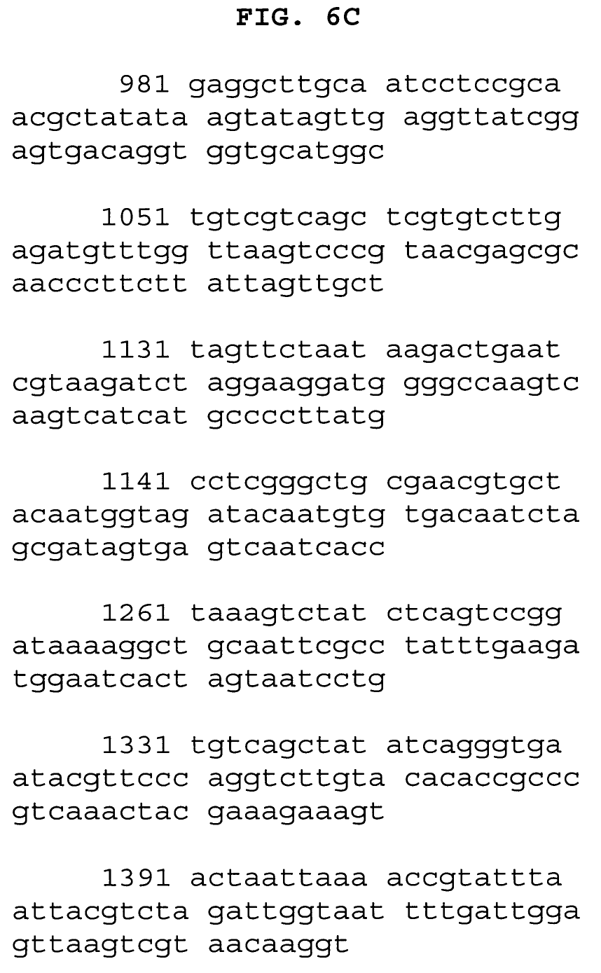

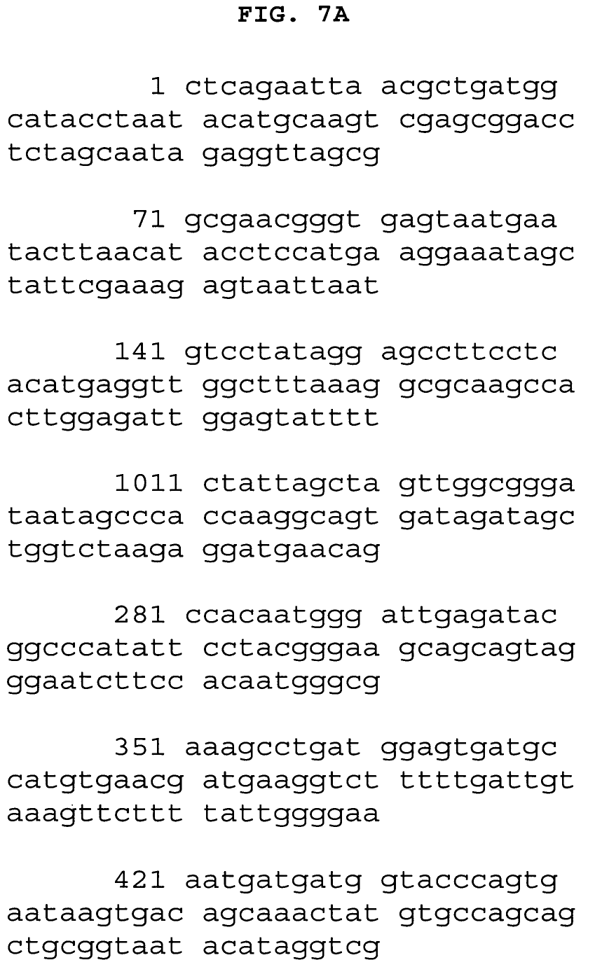

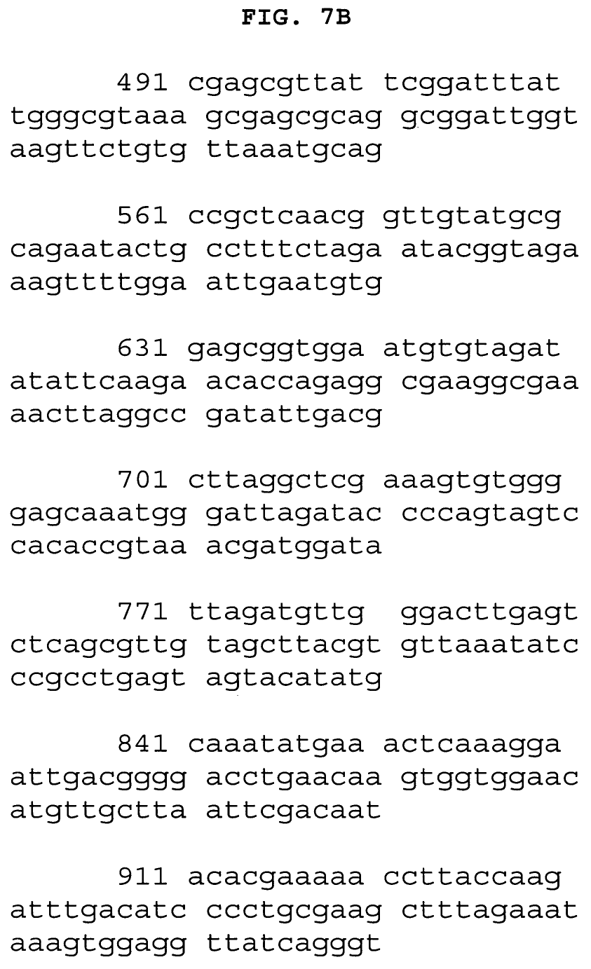

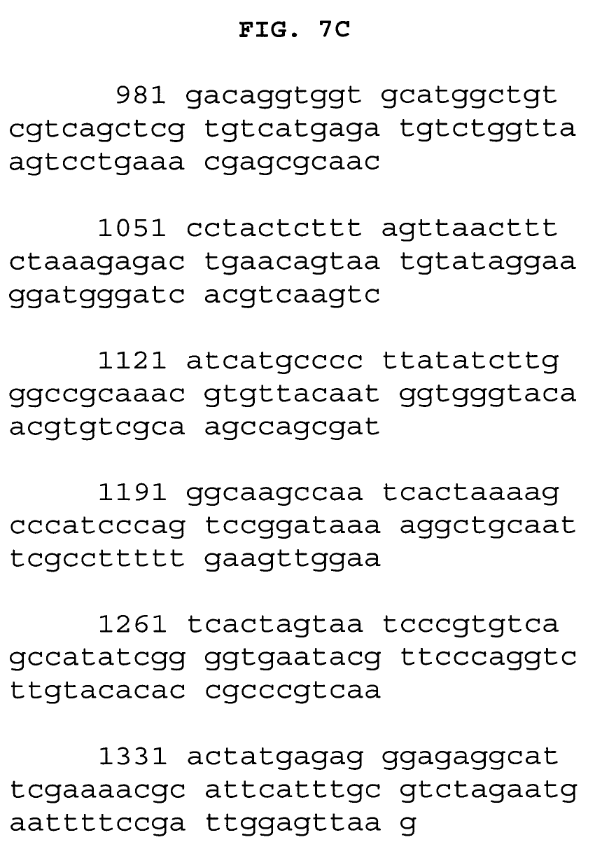

A partial sequence (417 nucleotides) of the DNA encoding 16S RNA from Mycoplasma haemosapiens is shown in FIG. 1 (SEQ ID NO:1) herein. The sequences of DNA encoding 16S RNA for Mycoplasma haemocanis (FIG. 4), Mycoplasma haemofelis (FIG. 5), Mycoplasma heamosuis (FIG. 6), and Mycoplasma haemomuris (FIG. 7) are also provided herein. Using one of the above sequences, a worker of ordinary skill in the art can utilize well known methods of detecting the presence of this RNA sequence, its genomic DNA equivalent or complementary sequences in a human body sample.

A. Detection of M. haemosapiens and lupus 16S rRNA Nucleic Acid

Using a nucleic acid probe, a person of ordinary skill in the art can determine the presence of all or a portion of the Mycoplasma haemosapiens 16S polynucleotide in a human body sample, and thereby the presence of the microbe itself and thereby the disease. The presence of a portion of the nucleotide sequence, ranging from a sequence of as few as about 10 nucleotides to the full sequence, and preferably about 15 to about 20 nucleotides of Mycoplasma haemosapiens 16S polynucleotide can be determined. Preferably, the nucleic acid probe has a sequence of about 10 to about 100 nucleotides in length. More preferably, the nucleic acid probe is about 15 nucleotides to about 20 nucleotides in length. Most preferably, the nucleic acid probe has the nucleotide sequence of SEQ ID NO:1. A nucleotide sequence complementary to such a DNA sequence or a nucleotide sequence that hybridizes with that DNA sequence or its complementary sequence is, of course, also contemplated.

There are many methods known in the art for the detection of specific nucleic acid sequences and new methods are continually reported. A great majority of the known specific nucleic acid detection methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope such as ³²P, ³⁵S, 90Y, ¹¹¹In, and ¹³¹I, a fluorescent dye such as FITC, AMC, dansyl chloride, eosin isothiocyanate, fluorescamine, TRITC or the other well known materials listed in the 1995 Sigma Chemical Co. or other catalogue, digoxygenin, biotin, an enzyme such as horseradish peroxidase, jack bean urease or alkaline phosphatase or an acridinium ester.

Another type of process for the specific detection of nucleic acids of exogenous organisms in a body sample known in the art are the hybridization methods as exemplified by U.S. Pat. No. 6,159,693 and No. 6,270,974, and related patents. To briefly summarize one of those methods, a nucleic acid probe of at least 10 nucleotides, preferably at least 15 nucleotides, having a sequence complementary to the Mycoplasma 16S polynucleotide sequence is hybridized in a body sample, subjected to depolymerizing conditions, and the sample is treated with an ATP/luciferase system, which will luminesce if the Mycoplasma 16S polynucleotide sequence is present.

A further process for the detection of hybridized nucleic acid takes advantage of the polymerase chain reaction (PCR). The PCR process is well known in the art (U.S. Pat. No. 4,683,195, No. 4,683,202, and No. 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed before. Most preferably the detection of the duplex is done using primers of SEQ ID NOs: 2 and 3 in PCR.

In yet another embodiment of PCR, the detection of the hybridized duplex comprises electrophoretic gel separation followed by dye-based visualization.

Fluorescence techniques are also known for the detection of nucleic acid hybrids. U.S. Pat. No. 5,691,146 describes the use of fluorescent hybridization probes that are fluorescence-quenched unless they are hybridized to the target nucleic acid sequence. U.S. Pat. No. 5,723,591 describes fluorescent hybridization probes that are fluorescence-quenched until hybridized to the target nucleic acid sequence, or until the probe is digested. Such techniques provide information about hybridization, and are of varying degrees of usefulness for the determination of single base variances in sequences.

Besides PCR, another embodiment is detection of hybridization to a duplex form by fluorescence resonance energy transfer. Some fluorescence techniques involve digestion of a nucleic acid hybrid in a 5′→3′ direction to release a fluorescent signal from proximity to a fluorescence quencher, for example, TaqMan (Perkin Elmer; U.S. Pat. No. 5,691,146 and No. 5,876,930) utilizes the 5′ exonuclease activity of thermostable polymerases such as Taq to cleave dual-labeled probes present in the amplification reaction (Wittwer et al., Biotechniques, 22:130-138, 1997; Holland et al., Pro. Nat. Acad. Sci., 88:7276-7280, 1991). Although complementary to the PCR product, the probes used in this assay are distinct from the PCR primer and are dually-labeled with both a molecule capable of fluorescence and a molecule capable of quenching fluorescence. When the probes are intact, intramolecular quenching of the fluorescent signal within the DNA probe leads to little signal. When the fluorescent molecule is liberated by the exonuclease activity of Taq during amplification, the quenching is greatly reduced leading to increased fluorescent signal.

An additional form of real-time PCR also capitalizes on the intramolecular quenching of a fluorescent molecule by use of a tethered quenching moiety. The molecular beacon technology utilizes hairpin-shaped molecules with an internally-quenched fluorophore whose fluorescence is restored by binding to a DNA target of interest (Kramer et al., Nat. Biotechnol., 14:303-308, 1996). Increased binding of the molecular beacon probe to the accumulating PCR product can be used to specifically detect SNPs present in genomic DNA.

Another general fluorescent detection strategy used for detection of SNP in real time utilizes synthetic DNA segments known as hybridization probes in conjunction with a process known as fluorescence resonance energy transfer (FRET) (Wittwer et al., Biotechniques, 22:130-138, 1997; Bernard et al., Am. J. Pathol., 153:1055-1061, 1998). This technique relies on the independent binding of labeled DNA probes to the target sequence. The close approximation of the two probes on the target sequence increases resonance energy transfer from one probe to the other, leading to a unique fluorescence signal. Mismatches caused by SNPs that disrupt the binding of either of the probes can be used to detect mutant sequences present in a DNA sample.

A method used in medical applications, typically with cellular samples, is Fluorescence In Situ Hybridization (FISH). FISH methods are already known in the art and involve exciting fluorophore-labeled DNA and RNA by means of optical radiation of noncoherent light sources (lamps) or coherent light sources (lasers) and for detecting fluorescence in two or also three dimensions with suitable detectors [for example, see U.S. Pat. No. 5,792,610 and the citations therein]. For applications of the present invention, the labeling is preferably carried out by specifically binding fluorophores, which enable detection of small gene areas. The fluorophore is coupled to the desired DNA region by fluorescence in situ hybridization (FISH). For applications of the present invention, FISH is most preferably carried out with a 16S rRNA-specific probe in bone marrow, spleen, liver cells or samples from other appropriate tissues.

Besides DNA-based methods of detection for the presence of Mycoplasma haemosapiens, antibody methods can also be utilized in another aspect of the invention. Preferably, the presence of Mycoplasma haemosapiens in a human body sample is determined by contacting the human body sample with antibody raised to one or more of Mycoplasma haemocanis raised in dogs, Mycoplasma haemofelis raised in cats, Mycoplasma haemosuis raised in swine, and Haemobartonella muris raised in mice and determining whether an antibody recognition event occurs; i.e., specific antigen-antibody binding occurs. The occurrence of specific antibody binding indicates the presence of Mycoplasma haemosapiens in the human body sample. Specific antibody binding can be determined by using an ELISA format or any of the other well-known antibody-antigen interaction formats.

A similar assay can be carried out using antibodies from the human patient body sample such as blood, plasma or serum, to contact an antigen of one or more of Mycoplasma haemocanis, Mycoplasma haemofelis, Mycoplasma haemosuis, and Haemobartonella muris that is present as a separate antigen or is present in a body sample such as skin, joints, blood, lungs, kidneys, heart, brain, saliva, gastrointestinal tract, bone marrow, liver, and nervous system from one or more of dogs infected with Mycoplasma haemocanis, cats infected with Mycoplasma haemofelis, swine infected with Mycoplasma haemosuis, and mice infected with Haemobartonella muris. The noted Mycoplasma or Haemobartonella antigen with which patient antibodies to Mycoplasma haemosapiens specifically bind can be separated from the non-human body sample prior to the contacting step as is discussed hereinafter, or prepared synthetically using recombinant technology. As above, specific antibody binding indicates the presence of Mycoplasma haemosapiens infection in the human patient.

As is well known in carrying out and antibody-antigen assay, the antibodies and antigen are mixed to contact one with the other. The admixture so formed is maintained for a time period sufficient for the specific interaction (binding) to take place. The components are frequently rinsed or otherwise manipulated to separate materials that are not specifically bound, and the presence of specific binding is determined. These assay techniques are well known in the art, are carried out under conditions of time, temperature and pH value that are well known, and will not be gone into here because of that wide-spread knowledge. Again, specific antibody binding can be determined by using an ELISA format or any of the other well-known antibody-antigen interaction formats.

Thus, the skilled worker could use one or more of the before-described nucleic acid assays or the two antibody-antigen assays described immediately above.

Mycoplasma haemosapiens 16S rRNA Nucleic Acid Probe

A contemplated probe for use in a method of the present invention contains at least 10 nucleotides and is obtained from the sequence of DNA that encodes the 16S rRNA sequence shown in FIG. 1 (SEQ ID NO:1). That probe contains a Mycoplasma haemosapiens 16S rRNA sequence, a sequence complementary to that DNA sequence or a nucleotide sequence that hybridizes with that DNA sequence or its complementary sequence, with sequence lengths as discussed previously.

As used herein, the term “nucleic acid probe” refers to an oligonucleotide or polynucleotide that hybridizes to another nucleic acid of interest, which in this case is the Mycoplasma haemosapiens 16S nucleic acid, under appropriate conditions. A nucleic acid probe can occur as in a purified restriction digest or be produced synthetically, recombinantly or by PCR amplification. As used herein, the term “nucleic acid probe” refers to the oligonucleotide or polynucleotide used in a method of the present invention to hybridize to a genomic DNA, cDNA or RNA sequence of the Mycoplasma haemosapiens 16S nucleic acid. That same oligonucleotide is equally useful as a primer for polymerization in a PCR method.

As used herein, the terms “complementary” or “complementarity” are used in reference to nucleic acids (i.e., a sequence of nucleotides) related by the well-known base-pairing rules that A pairs with T (or T) and C pairs with G. For example, the sequence 5′-A-G-T-3′, is complementary to the sequence 3′-T-C-A-5′.

The term “hybridization” is used herein in reference to the pairing of complementary nucleic acid strands. Hybridization and the strength of hybridization (i.e., the strength of the association between nucleic acid strands) is impacted by many factors well known in the art including the degree of complementarity between the nucleic acids, stringency of the conditions involved affected by such conditions as the concentration of salts, the Tₘ (melting temperature) of the formed hybrid, the presence of other components (e.g., the presence or absence of polyethylene glycol), the molarity of the hybridizing strands and the G:C content of the nucleic acid strands.

As used herein, the term “stringency” is used in reference to the conditions of temperature, ionic strength, and the presence of other compounds, under which nucleic acid hybridizations are conducted. With “high stringency” conditions, nucleic acid base pairing occurs only between nucleic acid fragments that have a high frequency of complementary base sequences. Thus, conditions of “weak” or “low” stringency are often required when it is desired that nucleic acids that are not completely complementary to one another be hybridized or annealed together. The art knows well that numerous equivalent conditions can be employed to comprise low stringency conditions. The choice of hybridization conditions is generally evident to one skilled in the art and is usually be guided by the purpose of the hybridization, the type of hybridization (DNA-DNA, or DNA-RNA), and the level of desired relatedness between the sequences (Sambrook et al., 1989, Nucleic Acid Hybridization, A Practical Approach, IRL Press, Washington D.C., 1985).

The stability of nucleic acid duplexes is known to decrease with an increased number of mismatched bases, and further to be decreased to a greater or lesser degree depending on the relative positions of mismatches in the hybrid duplexes. Thus, the stringency of hybridization can be used to maximize or minimize stability of such duplexes. Hybridization stringency can be altered by: adjusting the temperature of hybridization; adjusting the percentage of helix destabilizing agents, such as formamide, in the hybridization mix; and adjusting the temperature and/or salt concentration of the wash solutions. For filter hybridizations, the final stringency of hybridizations often is determined by the salt concentration and/or temperature used for the post-hybridization washes. In general, the stringency of hybridization reaction itself can be reduced by reducing the percentage of formamide in the hybridization solution.

High stringency conditions, for example, utilize high temperature hybridization (e.g., 65° C. to 70° C.) in aqueous solution containing 4× to 6×SSC (1×SSC=0.15 M NaCl, 0.015 M sodium citrate) or 40 to 45° C. in 50% formamide combined with washes at high temperature (e.g. 5° C. to 25° C. below the Tₘ), in a solution having a low salt concentration (e.g., 0.1×SSC). Moderate stringency conditions typically utilize hybridization at a temperature about 500C to about 650C in 0.2 to 0.3 M NaCl, and washes at about 500C to about 550C in 0.2×SSC, 0.1% SDS. Low stringency conditions can utilize lower hybridization temperature (e.g. 35° C. to 45° C. in 20% to 50% formamide) with washes conducted at a low intermediate temperature (e.g. 40 to 55° C.) and in a wash solution having a higher salt concentration (e.g. 2× to 6×SSC). Moderate stringency conditions are preferred for use in conjunction with the disclosed polynucleotide molecules as probes to identify clones encoding nucleoside diphosphate kinases of the invention.

As used herein, the term “Tₘ” is used in reference to the “melting temperature”. The melting temperature is the temperature at which 50 percent of a population of double-stranded nucleic acid molecules becomes dissociated into single strands. The equation for calculating the Tₘ of nucleic acids is well known in the art. The Tₘ of a hybrid nucleic acid is often estimated using a formula adopted from hybridization assays in 1 M salt, and commonly used for calculating Tₘ for PCR primers: [(number of A+T)×2° C.+(number of G+C)×4° C.]. C. R. Newton et al. PCR, 2^{nd} Ed., Springer-Verlag, New York, p. 24 (1997). This formula was found to be inaccurate for primers longer that 20 nucleotides. Id. Other more sophisticated computations exist in the art that take structural as well as sequence characteristics into account for the calculation of Tₘ. A calculated Tₘ is merely an estimate; the optimum temperature is commonly determined empirically using methods that are well known to workers of ordinary skill in this art.

B. Visual Identification of Intra-Erythrocytic Bodies

As noted previously, the spleen is regulatory in removing nuclear remnants and other intra-erythrocytic particles from erythrocytes, and splenectomy is an infrequently used treatment for the thrombocytopenia seen in some patients with SLE. However, the splenectomy patient noted before was found to have parasitemias of erythrocytes with intra-erythrocytic phase contrast-visible refractile bodies in up to about 16 percent of her studied erythrocytes. The intra-erythrocytic bodies were found to be very similar to the animal hemotropic Mycoplasmas such as Mycoplasma haemofelis, Mycoplasma haemocanis and Haemobartonella muris.

As also noted previously, Howell-Jolly bodies are described as about 1μ in diameter in an eccentric position in the erythrocyte. In contrast, the bodies seen in bovine anaplasmosis and in the patients with SLE are approximately 0.5μ and have phase retractile characteristics that are not described in Howell-Jolly bodies. It is believed that the site of generation of the agents seen in most SLE patients' erythrocytes is in the myeloid precursors differentiating to megakaryocyte and erythrocyte precursors or other cells of the bone marrow. These agents are normally removed by the spleen, but in the splenectomized patient are constantly circulating until the erythrocyte is senescent and destroyed in the reticulo-endothelial system. These particles in the erythrocytes, or erythrocyte progenitors in the marrow appear to have heretofore escaped detection because they morphologically resemble the normal metamorphosis of the normoblast, but tend to be smaller than nuclear remnants.

The present studies show that Mycoplasma haemosapiens is the causative agent of systemic lupus erythematosus (SLE). The organism, whose gene that encodes the 16S rRNA sequence is disclosed herein (SEQ ID NO:1), was identified in human body samples from a patient previously diagnosed with SLE, as described below. Both of these patients had a greater amount of cells infected with organisms identified as Mycoplasma haemosapiens as a result of their earlier splenectomies as shown below.

Patient A.

A first blood sample was obtained from a 30-year old African American female patient who had been splenectomized and presented with the symptoms of systemic lupus erythematosus (SLE) in 1993. Examination of her blood indicated that about 16 percent of her erythrocytes contained exogenous bacterial structures or parasites that were stainable with giemsa and acridine orange and were retractile in phase contrast microscopy.

This splenectomized female patient with SLE had a large number of parasitized erythrocytes (about 16 percent). She had been on doxycycline with informed consent for 16 months with initial and continued improvement. Because she had enough intra-erythrocytic parasites to be counted, her course of treatment has been followed and electron microscopy was carried out. When examined in December 1995, she still had about 1.1 percent parasitized erythrocytes seen by giemsa staining and phase contrast microscopy. When questioned about her medical history and examined in 2004, she had less than 1 percent parasitemia, and this may have followed treatment with Cyclosporine A.

Similar exogenous bacterial structures or parasites have also been found in the erythrocytes of other SLE patients, albeit at a lower level of infection involving about 0.1 percent or less of the erythrocytes. A blood sample received from another splenectomized patient from Norway, designated as patient B, vida infra, discussed in more detail below evidenced parasitization of about 0.6 percent of the erythrocytes.

Inasmuch as the erythrocytes of the SLE patients examined have been found to contain these giemsa- and acridine orange-stainable exogenous bacterial structures or parasites, and such exogenous bacterial structures are not present in the erythrocytes of healthy patients or persons suffering from other diseases so far examined, it is believed that those parasites are the infective agent that causes SLE. The telltale structures, and inference of the presence of Mycoplasma haemosapiens, can be identified by their 16S rRNA sequences, as disclosed herein.

Patient B.

A second sample of blood from which the sequence of the genomic DNA was obtained was itself obtained from a Caucasian Norwegian Female, born in 1976. At age 14, she developed thrombocytopenia and purpura. This necessitated a splenectomy for presumed hypersplenism, because her marrow showed increased megakaryocytes. She was treated with steroid medications.

At age 15, she underwent a splenectomy, and at age 17, she was noted to have glomerulonephritis, generalized lymphadenopathey and “butterfly malar rash”. She also experienced arthralgia and periorbital edema.

An antinuclear antibody test was positive, and the Lupus anticoagulant was negative. She underwent a kidney biopsy that was diagnostic of Lupus nephritis.

At age 19, she had a myocardial infarction, with a large ventricular thrombus formation; she was treated with Cyclosporine A, and corticosteroids. At age 21, she developed an arterial thrombus of her right leg, and it resulted in a surgical amputation of the leg below the knee.

Despite her early thrombocytopenia, she was noted on peripheral smear to exhibit markedly increased platelets, suffered a myocardial infarction, and intra cardiac mural thrombus formation

Patient C.

Patient C is a 53 year old Caucasian female, who has suffered from systemic Lupus Erythematosus for many years. Her illness is characterized by arthritis, neutropenia, proximal phalangeal arthritis and deformity, a positive ANA, recurrent staphylococcal infection and Sjogren's syndrome. She is maintained on Plaquinil and prednisone. She underwent a bone marrow aspiration and biopsy in 1999 to investigate her persistent leucopenia. Her bone marrow when examined in March of 1999 revealed no abnormalities.

This marrow specimen was re-examined in 2004 after having been archived for four years. Multiple inclusions were seen in many of the megakaryocytes upon this re-examination. These inclusions appear to have been overlooked by the original observations of this marrow examination. These inclusions were approximately 0.4 micron to 0.8 micron in diameter and exhibited considerable variation in size. These findings suggested that the organisms were undergoing changes consistent with reproduction within the cytoplasm of the megakaryocyte.

In accordance with recognized methods of determining the presence of an infectious agent in human specimens, a study was devised to assay whether the same organism was identifiable in other patients with SLE and not in controls. Thus, blood was collected from patients with SLE and matched controls, and mixed with a small quantity of EDTA that had been filtered through a 0.2 micron filter. Of the SLE patients studied, almost all exhibited the parasitization (the presence of the cellular inclusions).

SLE affects all body tissues although the mechanism is unclear. It is believed that M. haemosapiens is an intracellular obligate parasite that cannot be cultured on artificial culture media. It is further believed that the organism uses the reproductive mechanisms of the bone marrow, including the stem cell, myeloid precursors, and lymphocyte precursors. The time of infection and site of the stem cell or progenitor cells of the marrow determines the type of activity and reproductive activity of these cells by reprogramming by the instructive action of an unknown substance. That unknown substance, possibly similar to GATA-1, elicited by the infecting agent, although not causing destruction of the cell, leads to a change in the reproductive activity of the cell(s), and a possible overproduction of the products of the cell, which by chance have been programmed to produce changes such as in B cell antibodies, or to various tissues or other coincident infecting agents. In this activity, progenitor cells are altered to reproduce or decrease the various cell lines they are programmed to become. Some of these cells are progenitors of erythrocytes, megakaryocytes, or lymphocytic progenitors of either B or T cells. All of these disruptions are descried in some patients with SLE

These organisms are apparently present in the majority of patients with SLE but have escaped detection and culturing by investigators over the years. In almost all SLE patients examined, this new organism parasitizes less than one percent of circulating erythrocytes. The methodology of examining human blood films is crude unless very careful technique is used. Both giemsa and Wright's stains must be filtered before each staining or the stain precipitate can be confused with intra-erythrocytic bodies.

In this methodology, the blood is examined on a Zeiss microscope through an objective with phase contrast optics. Each identified particle is viewed through a high power light field under oil immersion.

The exogenous intracellular structures of the unknown bacteria are observed in infected erythrocytes as blue gray bodies usually in the marginal position and are about 0.4 to about 0.5 micron in longest dimension, whereas Howell-Jolly bodies are larger and are not phase contrast refractile. Verification that the exogenous bacterial structures are not artifacts is made by switching to phase contrast microscopy without moving the stage. The blue gray structure under phase contrast appears as a doubly refractile structure in the same erythrocyte in the same location. These intra-erythrocyte structures can also be confused with Heinz bodies, Howell-Jolly bodies, nuclear remnants, or simply be overlooked if not carefully examined as above.

Acridine orange can also be used as a stain for the exogenous bacterial structures. In that case, the blood film is fixed with saline to which 10 volume percent formalin has been added. After complete fixation for 24 hours, the film is examined under indirect fluorescent microscopy. Intra-erythrocytic structures containing RNA fluoresce bright orange and are usually present in the marginal position within an erythrocyte.

The quantification of percentage of erythrocytes parasitized is made by counting ten fields within a square defined by an optically projected prism, determining the number of parasitized erythrocytes within the ten fields, and dividing the total number of parasitized erythrocytes by the total number X 100. This product was identified as the percentage of parasitized erythrocytes.

The standard method for staining of

Anaplasma marginale using giemsa stain was used here. One could not be sure of seeing an intra erythrocytic bacteria unless phase contrast microscopy was used to confirm each individual structure identified. Only after dual viewing, was the observed blue gray body accepted as a structure within the erythrocyte stroma. It is not possible to detect the bodies of M. haemosapiens in monocytes, platelets or lymphocytes because all of these cells have human DNA, and all DNA stains non-specifically stain human and bacterial DNA. Thus bacterial inclusions in these cells, even if present are not detectable by these methods.

Experimental infection of animals with Anaplasma is used for study of that entity, and veterinary hematologists have not relied on phase microscopy, because they see parasitemias of 50 to 80 percent, though the inventors have observed phase contrast refractile bodies in each parasitized erythrocyte with A. marginale noted to be infected. Some investigators in this field were unaware that the organisms could be positively identified more easily by phase contrast microscopy.

The clinical and laboratory examination of both of these patients described the diagnosis of systemic lupus erythematosis because of the criteria described in LaHita, Systemic Lupus Erythematosus, Churchill Livingston (New York, 1987).

Both patients A and B had positive anti-nuclear antibodies, proteinuria, thrombocytopenia, anti-dsDNA antibodies, a diagnostic Lupus malar rash, elevated immunoglobulin, lupus renal disease, anemia, and arthralgia. Only four of these major criteria are required to make the diagnosis of systemic lupus erythematosus. (Westly H. Reeves, Robert G. Lahita, in Systemic Lupus Erythematosus, Second Ed., Churchill Livingston, N.Y. 1992).

Patient B's blood was examined for intra-erythrocytic bodies in 1996, and she was found to have approximately 1 percent of her erythrocytes containing intra-erythrocytic inclusions consistent with small bacterial structures. After a period of treatment with clarithromycin and rifampin, these structures were observed to undergo degenerative changes suggesting anti-bacterial effect. The antibiotic treatment was stopped by her physician.

When the blood of this patient was examined in February 2003, the number of organisms was established as 3.9 percent of the erythrocytes. The organisms were identified as different from Howell-Jolly bodies, because they were doubly retractile under phase contrast microscopy. These doubly refractile bodies were seen in the same locus as the giemsa-staining bodies in a marginal position in the erythrocyte.

When her blood was examined in November of 2003, the number of intra-erythrocytic inclusions had decreased to 0.5 percent.

Upon examination by polymerase chain reaction using probes (SEQ ID NOs: 2 and 3) the blood repeatedly showed the presence of Mycoplasma haemosapiens. The blood of other patients with SLE did not show the same product of PCR, presumably because of small numbers of infected cells and the function of the spleen in removing infected erythrocytes.

The presence of intra-erythrocytic organisms is rare to very rare in most Lupus patients, but in splenectomized patients, they are readily found. This is because the function of the spleen is to perform the maintenance of quality control of erythrocytes in the red pulp by removal of senescent or damaged erythrocytes from the circulation. (Harrison's Principles of Internal Medicine, 15^{th} Ed., Braunwald, Fauci et al, McGraw, Hill Medical Publishing Division.)

Both patients A and B give evidence of a specific perturbation of two major systems of the bone marrow. First, they were observed to have alterations of percentages of intra-erythrocytic inclusions identified as bacteria like, and different from Howell-Jolly bodies. These inclusions varied in concentrations, and seemed to fluctuate with treatment of the patient. They also were observed to undergo deterioration when viewed by light microscopy after specific antibiotic treatment.

In addition, patient B had extremely variable activities of her platelets and their effects on her vascular system. She began with not enough platelets and thrombocytopenic purpura, and then progressed to thrombiocytosis, with marked increases of the number of circulating platelets. This progressed to life-threatening complications of hypercoagulability, including intracardiac mural thrombi, and peripheral emboliztion with loss of tissue in the brain, cardiac muscle, and her leg.

In a discussion of the use of a transcription factor and erythrocytes, Iwasaki and others writing in Immunity, 19:451-462 (2003) describe the commonality of the progenitor for both megakaryoctes and erythrocytes.

These two cellular systems are derived from the same progenitor, and patients A and B were found to have significant disturbances in numbers, and evidence of disease producing changes to both of these cell lines. Both erythrocytes and megakaryocytes, the producers of platelets come from the same progenitor cell.

It is believed that the site of infection caused these two cell lines to become deranged and escape the limitations of the body on its systems to maintain homeostasis of these cells. It is clear that the infection has entered and deranged this single progenitor cell, and continues to use this progenitor for its own reproduction. The bacterial bodies are directly observed in the erythrocytes, which are not removed until the normal senescence of erythrocytes of 120 days, because the spleen is absent. The infecting bodies cannot be seen in the platelets by DNA staining such as Giemsa, because human DNA is present in the platelets. Human DNA and bacterial DNA cannot be differentiated by DNA stain. Their presence is obvious because of their obvious proliferation and alterations of function observed in the target cells.

It is believed that in both patients A and B the infected erythrocyte-megakaryocyte progenitor cell is a major site of alteration of their platelet and erythrocyte function, though there may be alteration of function of T and/or B cells by infection of their progenitor cells, as suggested by pericarditis and arthritis.

It is further believed that these two patients taken together along with the reported perturbation of so many systems of the elements of blood in Lupus as reported in Lahita, R. G. Systemic Lupus Erythematosus, Churchill, Livingston, N.Y. (1987) page XXIX, that the bone marrow is the primary site of infection by Mycoplasma haemosapiens, and is capable of causing the entire syndrome without requiring any other infection of other tissues.

The finding of intracellular bodies resembling the infectious agent from the other Lupus case, and identified as morphologically similar to Mycoplasma haemosapiens in the intracellular matrix of megakaryocytes of the bone marrow of patient C, further identifies this agent as being a primary infection of the bone marrow. There appeared to be no vacuole enclosing the bodies seen within bone marrow cells, although a vacuole may have been obscured by other factors such as the method of preparation, and changes resulting from the varied cytoplasmic contents of the megakaryocytes. There was also the appearance of variation in the size of the inclusions. Megakaryocytes are not normally found in the peripheral circulation, but supply platelets through cytoplasmic insertion into the small blood vessels.

It is believed that the megakaryocytes function in patients with SLE as a growth site for propagation of the parasite, identified as M. haemosapiens using the mechanism of the megakaryocytic nucleus to supply necessary growth factor and enzymes to the parasite, which has a deficiency of these factors needed for reproduction. This is consistent with the biological potential of an obligate intracellular parasite that depends on the nucleus for its growth. This observation is also consistent with the finding of the organism enclosed in a vacuole in the peripheral circulation, because the megakaryocyte and the erythrocyte have a common progenitor cell. The circulating erythrocyte has no nucleus, and the parasite having taken up its existence in the erythrocyte, becomes inert, cannot divide, and is protected from body defenses by the erythrocyte membrane. The parasite only is exposed to body defenses upon senescence of the erythrocyte, and its absorption by the reticuloendothelial system, or activity of the spleen, which removes defective, pitted erythrocytes, or erythrocytes containing other particles. The spleen is often enlarged in SLE.

Although the invasion of other cells in the marrow has not yet been observed, infection of other cells and progenitors of lymphoid and myeloid series is expected and explains the multi-faceted pathogenic mechanisms of the disease state known as Systemic Lupus Erythematosus.

C. Antibody Methods of Detecting Mycoplasma haemosapiens

Antibody methods well known in the art for detection of Mycoplasma cells in body samples from other species are applicable to the detection of Mycoplasma haemosapiens in a human body sample.

For example with Mycoplasma haemofelis, an organism with extremely close DNA homology to Mycoplasma haemosapiens in a body sample from a cat, e.g. work by Joanne Messick and coworkers, Mycoplasma haemofelis will infect red blood cells. In analysis of blood samples from the infected cat, in about 11 days to about 14 days, fresh antibodies to Mycoplasma haemofelis are evident. Similarly derived antibodies can be obtained from M. haemocanis (dogs), M. haemosuis (swine), or Haemobartonella muris (mice).

The antibodies to Mycoplasma haemofelis from cats are useful for antibody cross-reactivity studies utilizing in vitro analysis of human body samples, using any of the standard antibody methods of the art. Antibodies to Mycoplasma haemocanis (dogs), M. haemosuis (swine), or Haemobartonella muris (mice) can similarly be utilized in in vitro studies of antibody cross-reactivity.

Illustratively, erythrocytes recovered from cats infected with Mycoplasma haemofelis are admixed with an anti-coagulating amount of aqueous EDTA. That admixture and contacting of the cells with EDTA causes the microbiologic bodies infecting the blood cells to disengage from the cells. Those bodies and their antigens can be separated from the erythrocytes by differential centrifugation or other well known means. The separated Mycoplasma haemofelis microbiologic bodies provide a preparation of the microbiologic bodies that is relatively purified as compared to the infected erythrocytes. The separated microbiologic bodies or their antigenic portions can be used as an antigen in antibody-antigen studies such as an ELISA assay using antibodies from a patient's body sample.

### EXAMPLE 1

### PCR-Based Protocol for Detection of 16S rRNA-Encoding Genes from Human Patients with Systemic Lupus Erythematosis

I. DNA Extraction

DNA_{ZOL} BD (Molecular Research Center, Inc.), was utilized for genomic DNA isolation from 0.5 ml of whole blood of both healthy control subjects and lupus patients. Quantification of DNA by absorption at 260 nm was followed by agarose gel electrophoresis for comparison of DNAs based on intactness of genomic DNAS.

II. Polymerase Chain Reaction (PCR)

1. PCR:

AccuPrime™ Taq DNA Polymerase System (Invitrogen Life Technologies, Catalog no. 12339-016) was used for the PCR reaction. Components of the AccuPrime™ System developed by Invitrogen included the following in either 25 μl or 50 μl reaction volumes as follows:

| | Reaction Volume | | |
|---|---|---|---|
| Component | | 25 μl | 50 μl |

| | | | | |
|---|---|---|---|---|
| 10× AccuPrime ™ PCR Buffer II^{#} | 2.5 | μl | 5.0 | μl |
| Forward Primer (10 μM)* | 0.5 | μl | 1.0 | μl |
| Reverse Primer (10 μM)* | 0.5 | μl | 1.0 | μl |
| Template DNA | 10 | pg | 200 | ng |
| AccuPrime ™ Taq DNA Polymerase | 0.5 | μl | 1.0 | μl |
| Filtered (0.22 ml) Sterile | to 25 | μl | to 50 | μl |
| Milli-QH₂O ™ | | | | |

^{#}10× PCR Buffer II contains: 200 mM Tris-HCl (pH 7.4), 500 mM KCl, 15 mM MgCl₂, 2 mM dGTP, 2 mM dATP, 2 mM dTTP, 2 mM cCTP, “thermostable AccuPrime ™ protein”, 10% glycerol and proprietary components from the supplier.
*Primer final concentration: 0.2 μM.

Selection of the AccuPrime™ System for use with genomic DNA extracted from the blood of systemic lupus erythematosis (SLE) patients followed PCR trials with a variety of commercially available Taq DNA polymerase/buffer systems. The Invitrogen AccuPrime™ system was selected for routine use based upon improved specificity in PCR product amplification, as determined experimentally and as described by Invitrogen.

2. Primers: MST Macromolecular Facility

Forward and reverse DNA primers were designed at Michigan State University (MSU) and were synthesized by the MSU Macromolecular Facility, near positions 949 and 1404 (E. coli numbering), respectively. The primers were designed to hybridize with relatively conserved 16S ribosomal DNA sequences of blood-borne Mycoplasma bacteria from Mycoplasma haemofelis, M. haemocanis, M. haemosuis and Haemobartonella muris. The primer DNA sequences are as follows:

| | |
|---|---|
| Forward primer: | |
| 5′-AAGTGGTGGAGCATGTTGC-3′ | SEQ ID NO:2 |
| Reverse primer: | |
| as | |
| 5′-TAGTTTGACGGGCGGTGTG-3′ | SEQ ID NO:3 |

Using primer concentrations provided in preparation by a final concentration of 0.20 μM for each of the primers, as specified in the AccuPrime™ protocol was utilized.

3. Thermal cycling conditions (Peltier™ Thermal Cycler, Model PTC-200, MJ Research):

| Step | Temperature (° C.) | Time | |
|---|---|---|---|
| 1 | 95 | 3 | minutes |
| 2 | 94 | 30 | seconds |
| 3 | 58 | 30 | seconds |
| 4 | 72 | 45 | seconds |
| 5 | repeat steps 2-4 for 39 cycles | | |
| 6 | 72 | 10 | minutes |

| | | |
|---|---|---|
| 7 | 4 | hold |

| Forward Primer: DNA Target For 1GS rRNA Sequence | | | |
|---|---|---|---|
| | EpeSuis | AACAAGTGGT GGAGCATGTT GCTTAATTCG | SEQ ID NO:4 |
| | HmbFeli | AACAAGTGGT GGAGCATGTT GCTTAATTCG | SEQ ID NO:5 |
| | HmbCani | AACAAGTGGT GGAGCATGTT GCTTAATTCG | SEQ ID NO:6 |
| | E. coli | CACAAGCGGT GGAGCATGTG GTTTAATTCG | SEQ ID NO:7 |
| | Primer | AAGTGGT GGAGCATGTT GC | SEQ ID NO:2 |
| Reverse Primer: DNA Target For 1GS rRNA Sequence | | | |
| | EpeSuis | GTGTTGTACA CACCGCCCGT CAAACTACGA | SEQ ID NO:8 |
| | HmbFeli2 | GTCTTGTACA CACCGCCCGT CAAACTATGA | SEQ ID NO:9 |
| | HmbCani2 | GTCTTGTACA CACCGCCCGT CAAACTATGA | SEQ ID NO:10 |
| | E. coli | GCCTTGTACA CACCGCCCGT CACACCATGG | SEQ ID NO:11 |
| | 172 | | |
| | Primer | TAGTTTG ACGGGCGGTG TG | SEQ ID NO:3 |

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A method for diagnosing systemic lupus erythematosus in a human patient that comprises detecting Mycoplasma haemosapiens in the patient, wherein said Mycoplasma haemosapiens is detected by one or more of (i) determining the presence of DNA that encodes all or part of the Mycoplasma haemosapiens 16S rRNA or a sequence complementary to that DNA in a human body sample, or (ii) contacting a human body sample from the patient with antibodies raised to one or more of Mycoplasma haemocanis in dogs, Mycoplasma haemofelis in cats, Mycoplasma haemosuis in swine, and Haemobartonella muris in mice and determining whether specific antibody binding occurs, or (iii) contacting antibodies from the human patient with a body sample from one or more of dogs infected with Mycoplasma haemocanis, cats infected with Mycoplasma haemofelis raised, swine infected with Mycoplasma haemosuis, and mice infected with Haemobartonella muris, and determining whether specific antibody binding occurs.

2. The method of claim 1 wherein the human body sample is selected from the group consisting of skin, joints, blood, lungs, kidneys, heart, brain, saliva, gastrointestinal tract, bone marrow, liver, and nervous system.

3. The method of claim 1 wherein the human body sample is blood.

4. The method of claim 1 wherein a nucleic acid probe of at least about ten nucleotides in length from a sequence present in one or more of the nucleic acids of SEQ ID NOs: 1-3 or 12-15 is utilized to determine by hybridization to a duplex form the presence of DNA that encodes Mycoplasma haemosapiens 16S rRNA.

5. The method of claim 4 wherein the detection of hybridization to the duplex form comprises a Southern blot technique.

6. The method of claim 4 wherein a nucleic acid probe used in the Southern blot has been labeled with a tag selected from the group consisting of a radioactive isotope, a fluorescent dye, digoxygenin, horseradish peroxidase, an alkaline phosphatase or an acridinium ester.

7. The method of claim 4 wherein the detection of the hybridization to a duplex form comprises an ATP/luciferase system.

8. The method of claim 4 wherein the detection of hybridization to a duplex form comprises a polymerase chain reaction.

9. The method of claim 8 wherein the nucleic acid probe is labeled with a tag selected from the group consisting of a radioactive isotope, a fluorescent dye, digoxygenin, horseradish peroxidase, alkaline phosphatase, an acridinium ester, biotin and jack bean urease.

10. The method of claim 8 wherein a method of detection of the hybridized duplex comprises electrophoretic gel separation followed by dye-based visualization.

11. The method of claim 4 wherein the hybridization to a duplex form is detected by fluorescence resonance energy transfer.

12. The method of claim 4 wherein a thermostable polymerase with exonuclease activity and dually-labeled probes with both a molecule capable of fluorescence and a molecule capable of quenching fluorescence are used in fluorescence resonance energy transfer.

13. The method of claim 8 comprising real-time PCR and a hairpin-shaped probe with an internally-quenched fluorophore.

14. The method of claim 4 wherein detection comprises fluorescence in situ hybridization (FISH).

15. The method of claim 4 wherein the nucleic acid probe has a nucleotide sequence comprised of SEQ ID NO:8.

16. The method of claim 4 wherein said nucleic acid probe is about 10 nucleotides to about 100 nucleotides in length.

17. The method of claim 4 wherein said nucleic acid probe is about 15 nucleotides to about 20 nucleotides in length.

18. The method of claim 1 wherein the detecting comprises using primers of SEQ ID NOs: 2 and 3 in a polymerase chain reaction.

19. The method of claim 1 wherein the detecting comprises contacting a human body sample from the patient with antibodies raised to one or more of Mycoplasma haemocanis in dogs, Mycoplasma haemofelis in cats, Mycoplasma haemosuis in swine, and Haemobartonella muris in mice, and determining whether specific antibody binding occurs.

20. A method of detecting the presence of Mycoplasma haemosapiens in a human body sample comprising the step of contacting the human body sample with antibodies raised to Mycoplasma haemocanis in dogs, Mycoplasma haemofelis in cats, or Mycoplasma haemosuis in swine, or Haemobartonella muris in mice and determining whether specific antibody binding occurs.

21. The method according to claim 20 wherein the determination of whether specific antibody binding occurs is carried out using an ELISA format.

22. A method of detecting the presence of Mycoplasma haemosapiens in a human patient comprising the step of contacting antibodies from the human patient with a body sample from one or more of dogs infected with Mycoplasma haemocanis, cats infected with Mycoplasma haemofelis raised, swine infected with Mycoplasma haemosuis, and mice infected with Haemobartonella muris, and determining whether specific antibody binding occurs.

23. The method according to claim 22 wherein the determination of whether specific antibody binding occurs is carried out using an ELISA format.


SEQUENCE LISTING (15 sequences)

SEQ ID NO: 1 - DNA, 417 bp, Mycoplasma haemosapiens
ttaattcgataatacacgaaaaaccttaccaaggtttgacatccctcgcaaagctatagaaatatagtagaggttatcgaggtgtcaggtggtgcatggctgtcgtcagctcgtgtcttgagatgtttggttaagtcccgcaacgagcgcaaccccactctttagttacttgtctaaagagactgcacagtaatgtagaggaaggatgggatcacgtcaagtcatcatgccccttatgccttgggctgcaaacgtgctacaatggcgaacacaatgtgttgcaaaccagcgatggtaagctaatcaccaaatttcgtctcagttcggataggaggctgcaattcgcctccttgaagttggaatcactagtaatcccgtgtcagctatatcggggtgaatccgttcccaggtcttgta

SEQ ID NO: 2 - DNA, 19 bp, Artificial sequence
  forward primer
aagtggtggagcatgttgc

SEQ ID NO: 3 - DNA, 19 bp, Artificial sequence
  Reverse primer
tagtttgacgggcggtgtg

SEQ ID NO: 4 - DNA, 30 bp, Artificial sequence
  Forward primer for EpeSuis
aacaagtggtggagcatgttgcttaattcg

SEQ ID NO: 5 - DNA, 30 bp, Artificial sequence; identical to SEQ ID NO: 4
  HmbFeli forward primer

SEQ ID NO: 6 - DNA, 30 bp, Artificial sequence; identical to SEQ ID NO: 4
  HmbCani forward primer

SEQ ID NO: 7 - DNA, 30 bp, E. coli; identical to SEQ ID NO: 4

SEQ ID NO: 8 - DNA, 30 bp, Artificial sequence
  Reverse primer for EpeSuis
gtgttgtacacaccgcccgtcaaactacga

SEQ ID NO: 9 - DNA, 30 bp, Artificial sequence
  Reverse primer for HmbFeli2
gtcttgtacacaccgcccgtcaaactatga

SEQ ID NO: 10 - DNA, 30 bp, Artificial sequence; identical to SEQ ID NO: 9
  Reverse primer for HmbCani2

SEQ ID NO: 11 - skipped

SEQ ID NO: 12 - DNA, 1393 bp, Mycoplasma haemocanis
aattaatgctgatggtatgcctaatacatgcaagtcgaacggaccttggtttcggccaaggttagtggcaaacgggtgagtaatacatatctaacatgcccctctgtgggggatagccacttgaaaaagtgattaataccccataggaagctttatccatgatttagcttttaaagccttcgggcgctgagggattgggatatgctctattagctagttggcgggataaaagcccaccaaggcaatgatagatagctggtcttagaggatgaacagccacaatgggattgagatacggcccatattcctacgggaagcagcagtagggaatcttccacaatggacgaaagtctgatggagcaataccatgtgaacgatgaaggcctttttggttgtaaagttcttttacgagggataattatgatagtacttcgtgaataagtgacagcaaactatgtgccagcagctgcggtaatacataggtcgcaagcattattcggatttattgggcgtaaagcaagcgcaggcggatgtgtaagttctgtgttaaatgcagctactcaatagttgtatgcaccgaatactacatgtctagattgtggtagggagtttcggaattaagcatggagcggtggaatgtgtagatatgcttaagaacaccagaggcgaaggcggaaacttaggccataaatgacgcttaggcttgaaagtgtggggagcaaatgggattagataccccagtagtccacaccgtaaacgatgggtattagatattagggctttagctttagtgttgtagcttacgcgttaaataccccgcctgggtagtacatatgcaaatatgaaactcaaaggaattgacggggacctgaacaagtggtggagcatgttgcttaattcgataatacacgaaaaaccttaccaaggtttgacatccctcgcaaagctatagaaatatagtagaggttatcgaggtgacaggtggtgcatggctgtcgtcagctcgtgtcttgagatgtttggttaagtcccgcaacgagcgcaaccccactctttagttacttgtctaaagagactgcacagtaatgtagaggaaggatgggatcacgtcaagtcatcatgccccttatgccttgggctgcaaacgtgctacaatggcgaacacaatgtgttgcaaaccagcgatggtaagctaatcaccaaatttcgtctcagttcggataggaggctgcaattcgcctccttgaagttggaatcactagtaatcccgtgtcagctatatcggggtgaatccgttcccaggtcttgtacacaccgcccgtcaaactatgagaggagtgggcatttaaaaatacatttatttgtatctagagtgaacattctgattggagtt

SEQ ID NO: 13 - DNA, 1396 bp, Mycoplasma haemofelis DNA
cagaattaatgctgatggtatgcctaatacatgcaagtcgaacggactttggtttcggccaaggttagtggcaaacgggtgagtaatatatatctaacatgcccctctgtgggggatagccacttgaaaaagtgattaataccccataggaagctttatccatgatttagcttttaaagccttcgggcgctgagggattgggatatgctctattagctagttggcgggataaaagcccaccaaggcaatgatagatagctggtcttagaggatgaacagccacaatgggattgagatacggcccatattcctacgggaagcagcagtagggaatcttccacaatggacgaaagtctgatggagcaataccatgtgaacgatgaaggcctttttggttgtaaagttcttttacgagggataattatgatagtacttcgtgaataagtgacagcaaactatgtgccagcagctgcggtaatacataggtcgcgagcattattcggatttattgggcgtaaagcaagcgcaggcggatgtgtaagttctgtgttaaatgcagctactcaatagttgtatgcaccgaatactacatgtctagattgtggtagggagtttcggaattaagcatggagcggtggaatgtgtagatatgcttaagaacaccagaggcgaaggcggaaacttaggccataaatgacgcttaggcttgaaagtgtggggagcaaatgggattagataccccagtagtccacaccgtaaacgatgggtattagatattagggctttagctttagtgttgtagcttacgcgttaaataccccgcctgggtagtacatatgcaaatatgaaactcaaaggaattgacggggatctgaacaagtggtggagcatgttgcttaattcgataatacacgaaaaaccttaccaaggtttgacatccctcgcaaagctatagaaatatagtagaggttatcgaggtgacaggtggtgcatggctgtcgtcagctcgtgtcttgagatgtttggttaagtcccgcaacgagcgcaaccccactctttagttacttgtctaaagagactgcacagtaatgtagaggaaggatgggatcacgtcaagtcatcatgccccttatgccttgggctgcaaacgtgctacaatggcgaacacaatgtgttgcaaaccagcgatggtaagctaatcaccaaatttcgtctcagttcggataggaggctgcaattcgcctccttgaagttggaatcactagtaatcccgtgtcagctatatcggggtgaatccgttcccaggtcttgtacacaccgcccgtcaaactatgagaggagtgggcatttaaaaatacattcatttgtatctagagtgaacattctgattggagtt

SEQ ID NO: 14 - DNA, 1468 bp, Mycoplasma DNA
agagtttgatcctggctcaggattaatgctggtggtatgcataacacatgcaagtcgaacgaaaaaggtcttcgagcctttttagtggcaaacgggcgagtaacacatatttaacttgctcatccgaggagaatagcagcccgaaagggctattaatacgccatagttttaaattagtgaattaatttaaattaaaggaggctgccgaaaggtggcctcgcggatgaataggaatatgtcctattaggtcgttggagaggtaatggctcaccaagccgatgatgggtagctggactgagaggttgaacagccgcaatgggattgagaaatggcccatattcctacgggaagcagcagtgaggaatttttcacaatggacgaaagtctgatggagcaataccacgtgaacgatgaaggtcttctgattgtaaagttcttttatttaggaaaaaaagcgcggcaggaaatggccgcgccttgattgtactaattgaataagtgacagctaactatgtgccagcagctgcggtaaaacataggtcacgagcattatccggatttattgggcgtaaaggaagcgtaggctgaaatgtgtattcattgttaaaaatatttgcttaacaagtgttcgcggtgaagattacatttctagaattagttagagggtactggaattcaatgtgtagtggtggaatacgtagatatattgaggaacaccagaggctaaggcgagtgcctggaacataattgacgctgaggcttgaaagcgtgggtagcaaatgggattagataccccagtagtccacgccgtaaacgatgggtattagtcatttggatttaagactgagtgatgtagctaacgcgttaaataccccgcctgggtagtatatatgcaaatatgaaactcaaagaaattgacggggacctgaacaagtggtggagcatgttgcttaattcgataatacacgcaaaaccttaccgaggcttgcaatcctccgcaacgctatataagtatagttgaggttatcggagtgacaggtggtgcatggctgtcgtcagctcgtgtcttgagatgtttggttaagtcccgtaacgagcgcaacccttcttattagttgcttagttctaataagactgaatcgtaagatctaggaaggatggggccaagtcaagtcatcatgccccttatgcctcgggctgcgaacgtgctacaatggtagatacaatgtgtgacaatctagcgatagtgagtcaatcacctaaagtctatctcagtccggataaaaggctgcaattcgcctatttgaagatggaatcactagtaatcctgtgtcagctatatcagggtgaatacgttcccaggtcttgtacacaccgcccgtcaaactacgaaagaaagtactaattaaaaccgtatttaattacgtctagattggtaattttgattggagttaagtcgtaacaaggt

SEQ ID NO: 15 - DNA, 1401 bp, Mycoplasma haemomuris
ctcagaattaacgctgatggcatacctaatacatgcaagtcgagcggacctctagcaatagaggttagcggcgaacgggtgagtaatgaatacttaacatacctccatgaaggaaatagctattcgaaagagtaattaatgtcctataggagccttcctcacatgaggttggctttaaaggcgcaagccacttggagattggagtattttctattagctagttggcgggataatagcccaccaaggcagtgatagatagctggtctaagaggatgaacagccacaatgggattgagatacggcccatattcctacgggaagcagcagtagggaatcttccacaatgggcgaaagcctgatggagtgatgccatgtgaacgatgaaggtctttttgattgtaaagttcttttattggggaaaatgatgatggtacccagtgaataagtgacagcaaactatgtgccagcagctgcggtaatacataggtcgcgagcgttattcggatttattgggcgtaaagcgagcgcaggcggattggtaagttctgtgttaaatgcagccgctcaacggttgtatgcgcagaatactgcctttctagaatacggtagaaagttttggaattgaatgtggagcggtggaatgtgtagatatattcaagaacaccagaggcgaaggcgaaaacttaggccgatattgacgcttaggctcgaaagtgtggggagcaaatgggattagataccccagtagtccacaccgtaaacgatggatattagatgttgggacttgagtctcagcgttgtagcttacgtgttaaatatcccgcctgagtagtacatatgcaaatatgaaactcaaaggaattgacggggacctgaacaagtggtggaacatgttgcttaattcgacaatacacgaaaaaccttaccaagatttgacatcccctgcgaagctttagaaataaagtggaggttatcagggtgacaggtggtgcatggctgtcgtcagctcgtgtcatgagatgtctggttaagtcctgaaacgagcgcaaccctactctttagttaactttctaaagagactgaacagtaatgtataggaaggatgggatcacgtcaagtcatcatgccccttatatcttgggccgcaaacgtgttacaatggtgggtacaacgtgtcgcaagccagcgatggcaagccaatcactaaaagcccatcccagtccggataaaaggctgcaattcgcctttttgaagttggaatcactagtaatcccgtgtcagccatatcggggtgaatacgttcccaggtcttgtacacaccgcccgtcaaactatgagagggagaggcattcgaaaacgcattcatttgcgtctagaatgaattttccgattggagttaag